(19) United States
(12) Patent Application Publication
(10) Pub. No.: US 2008/0275356 A1
(43) Pub. Date: Nov. 6, 2008

(54) RESPIRATORY SENSING BELT USING PIEZO FILM

(76) Inventors: Peter Stasz, St. Paul, MN (US); Alan B. Jones, Maple Grove, MN (US)

Correspondence Address: NIKOLAI & MERSEREAU, P.A., 900 SECOND AVENUE SOUTH, SUITE 820, MINNEAPOLIS, MN, 55402, US

(21) Appl. No.: 11/743,839

(22) Filed: May 3, 2007

(51) Int. Cl. A61B 5/08

(52) U.S. Cl. 600/534

(57) ABSTRACT

A respiratory belt sensing device to aid in monitoring breathing patterns of a user, particularly in diagnosis and treatment of persons with breathing disorders. The device includes a PVDF film member having first and second metalized major surfaces and a perimeter containing a plurality of spaced apart alignment features. It also includes a first tabbed lead and a second tabbed lead respectively attached to the first and second major surfaces of the PVDF film member by an electrically conductive adhesive. The resulting assembly is affixed to the surface of a body encircling belt member. Signals are generated when the PVDF film on the belt member is stressed due to stretching as a user breathes in and out. The generated signals can thereafter be used in diagnosis and analysis.

### BACKGROUND OF THE INVENTION

I. Field of the Invention

This invention relates generally to a respiratory sensing belt which incorporates a piezoelectric film as its sensing element, and more particularly to a uniquely respiratory sensing belt design in which a piezoelectric film is subjected primarily to a stretching stress to generate data.

II. Discussion of the Prior Art

For many years, devices have been sought and developed to help monitor breathing patterns of human beings in an effective way. This is especially true with respect to respiratory sensors in sleep labs which monitor persons with breathing disorders such as sleep apnea. When using this type of sleep monitoring equipment it is well known that it is very important to have an accurate and reliable way to detect breathing variations.

In the past, a variety of materials and methods have been used to detect respiratory patterns. Some of these devices used film transducers such as polyvinylidene fluoride (PVDF) as a sensing element. PVDF is a highly non-reactive and pure fluoropolymer also known under the commercial name KYNAR®. PVDF material may exhibit efficient piezoelectric and pyroelectric properties. PVDF is electrically poled before it exhibits piezo and pyroelectric properties. It has been used in many types of sensor and battery applications. There are several examples of past respiratory sensing devices which use PVDF film. One example utilized a piece of PVDF film affixed proximate a person's airway so that air will impinge upon the film when respiratory gas is inspired or expired. An output signal was thereby produced related to the impingement of respiratory air on the sensor due to temperature changes or vibrations due to snoring.

Other ways of detecting respiration included using devices with belt and transducer arrangements. Such devices used a transducer arrangement where piezoelectric materials were mounted on belts located around a person's chest or abdomen. The sensors coupled to the belt could detect expansion and contraction to produce output signals. These signals would then be used to relay information about a subject's breathing for analysis. A limitation found in many of these devices was that flexing or bending of the PVDF material was required for signal detection. Therefore, if the individual wearing such a respiratory sensing belt was lying on the portion of the belt where the PVDF material was located, the necessary bending and signal generation would not occur and any previously generated signal would effectively disappear.

Further, the attachment arrangements used in many of the past devices were not particularly well suited for measuring a stretching stress on a belt or like device. Likewise, the configuration used was not well suited for manufacture and the PVDF material proved difficult to work with for these designs. Also, the shape of the PVDF or other sensing element was not ideally shaped to detect longitudinal stress and stretching. Because the PVDF is strictly used to measure changes in stress, the amplitude of the signal changes very little when a person lies on the sensor or when the belt is looser or tighter and there is no signal inversion, as exists with the belts using bending of the sensor as a signal generation.

Therefore, it is desirable to have a sensing device that can be used with greater versatility, ease of manufacture, and effectiveness. An improved respiratory belt sensing device is needed which overcomes the problems experienced in past methods and devices.

### SUMMARY OF THE INVENTION

The present invention relates generally to a respiratory sensing belt. The device includes a film member having piezoelectric properties and first and second metalized major surfaces. The surfaces have a symmetric shape and a plurality of alignment features around their perimeter. The invention also includes a first tabbed lead and a second tabbed lead, respectively attached to the first and second major surfaces of the film member by an electrically conductive adhesive. This assembly may then be sandwiched between thin layers of an elastomeric material such as polyurethane, which is capable of being stretched. A label is used to conceal the film member and assembly which is affixed to the surface of an elastic belt member.

The foregoing features, objects and advantages of the invention will become apparent to those skilled in the art from the following detailed description of a preferred embodiment, especially when considered in conjunction with the accompanying drawings.

### DESCRIPTION OF THE DRAWINGS

FIG. 1 is an exploded view of the respiratory sensing belt of the present invention;

FIG. 2 is a top perspective view of the piezoelectric film member;

FIG. 3 is a top view of the respiratory sensing belt in its assembled configuration; and

FIG. 4 is an exploded view of an alternate embodiment of the respiratory sensing belt.

### DESCRIPTION OF THE PREFERRED EMBODIMENT

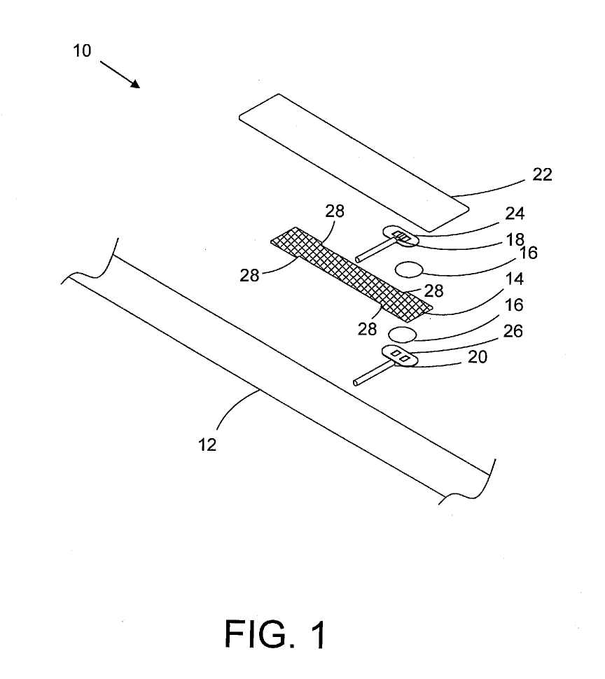

The present invention can be readily understood from FIGS. 1-4. As shown in the exploded view in FIG. 1, the belt sensing device generally includes a sensor member assembly 10 and a belt member 12. The sensor member includes a PVDF film member 14, two pieces of electrically conductive double sided tape 16, first and second tabbed wire leads 18 and 20, and a label 22. These components are assembled as a single uniform sensor member 10. When assembled, this sensor member 10 will be attached to the outside surface of a belt member 12 such that when the belt is stretched an output signal is generated from the PVDF film. These signals are received by tabbed wire leads 18 and 20, and passed to circuitry allowing for analysis of the signals transmitted. This configuration and operation will be set forth in the following description more fully.

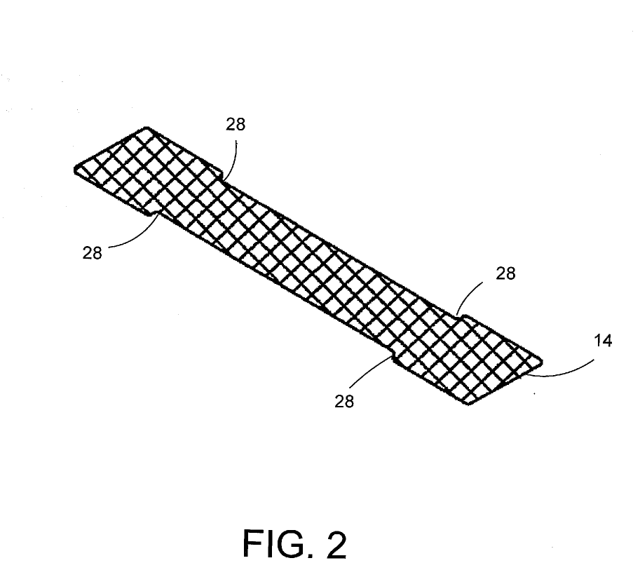

The configuration of the film member 14 is important to the capabilities and manufacturability of the sensor assembly. While this film member is generally PVDF film, a similar piezoelectric film material of related properties may be utilized as well. The PVDF film 14 is a single-piece, flat member having an outline of a central rectangular member with two rectangular ends of slightly larger width. This outline can be described as a “dogbone” or “dumbbell” shape. The corners of the film are rounded, containing a slight radius. A more detailed view of PVDF film member 14 can be seen in FIG. 2. This film is metalized on both major surfaces. The elongated rectangular shape of the member provides a configuration which can easily detect forces exerted due to stress experienced by the PVDF film 14 in the longitudinal direction along its length. The enlarged head provides a location where signals produced along the film's length can be concentrated and sensed by tabbed wire leads 18 and 20. The symmetrical shape of the PVDF member allows for easy manufacture and assembly as the film may be readily reversed or inverted during such operations. Typically, the entire length of the PVDF member 14 is only a few inches long and the width is only a fraction of an inch across.

As mentioned above, the perimeter of PVDF member 14 is somewhat dumbbell shaped, having a central rectangular section and two enlarged heads at the ends. Four important alignment points 28 are located where the perimeter of the central rectangular section and the perimeter of the enlarged heads come together. In this embodiment, these alignment points 28 not only serve to properly orient the surrounding sensor structure, but also function as locations at which adhesive is applied to hold the PVDF film 14 against the belt member 12.

Looking again at the exploded view of FIG. 1, the relative locations of the conductive members 16 and tabbed wires 18 and 20 in the sensor assembly can be understood. The two tabbed wires 18 and 20 are found above and below the centrally located PVDF film member 14 at one of the enlarged heads of the film. The assembly surrounding tabbed wire lead 18, located above the top major surface of the PVDF member, is aligned such that the tab 24 extends longitudinally across the enlarged head of the PVDF member. Accordingly, the leads 18 of the tabbed wire protrude from the sensor body 10 in a direction perpendicular to the 24 of the tabbed wire. Similarly, the lower tabbed wire leads 20 and base 26 are situated in a mirrored configuration on the opposing face of the PVDF film member. It is important to note that the tabbed wires are positioned such that no portion of the tabbed wire 18 contacting the upper major surface of the PVDF film 14 is in contact with the tabbed wire 20 of the lower major surface of the PVDF film 14.

Located directly between the tabs 24 and 26 of the tabbed wires and the PVDF film member 14 are pieces of a double-sided, electrically-conductive adhesive tape members 16. One such material is also known under the commercial name ARCLAD®. These ARCLAD pieces 16, like the tabbed wire members 18 and 20, do not make contact with the opposing surfaces of the PVDF film 14 or extend beyond the perimeter of the PVDF film.

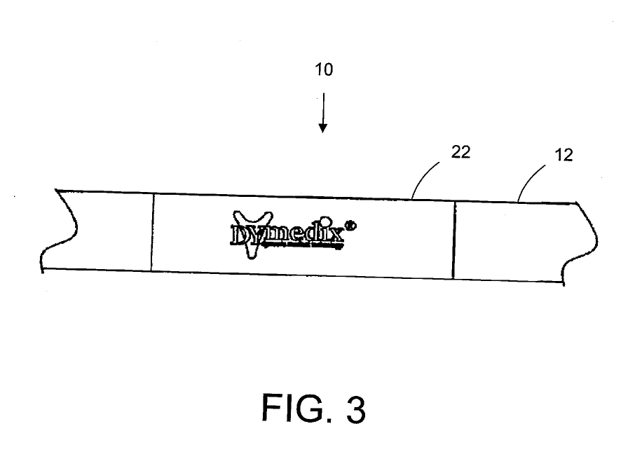

Finally, the label 22 is placed over the entire sensor member assembly and joined to the belt 12. This is done by applying an adhesive to the bottom face of the label 22 and placing it in alignment with the outer dimensions of the belt 12. Label 22 may also be made from stretchable material. The final assembled configuration appears as shown in FIG. 3. In FIG. 3, the entire sensor assembly is covered by the label 22 in this top view. The belt 12 is generally made of an elasticized nylon material that allows for stretching its length in the longitudinal direction.

Once assembled, the device of the present invention generally operates as follows. First, a user places the belt comprising the present invention around his or her chest or abdomen. Next, when the user inhales air and thereby expands his or her chest cavity or abdomen, tension is applied at opposed ends of the PVDF transducer assembly mounted to the belt. Similarly, when a user exhales air, tension is reduced producing a signal reflecting decreased tension. An electrical signal proportional to the tension stress is produced across the tabbed wires 18 and 20. These wires extend to amplifying and wave shaping circuitry which can be used by medical personal to diagnose and analyze the respiratory patterns of the user.

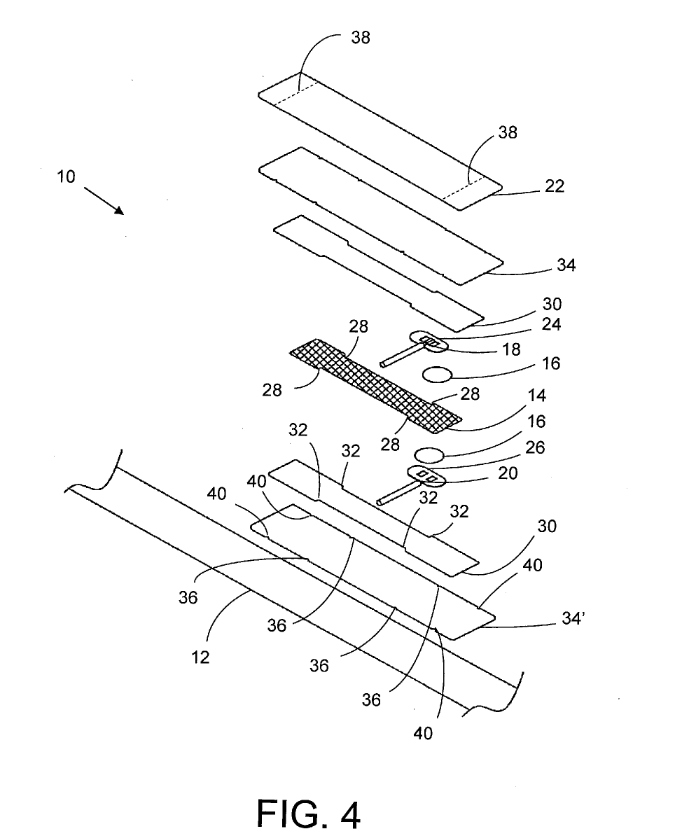

An alternate embodiment of the present invention is set forth in the exploded view of FIG. 4. In this embodiment, the PVDF film member 14 is centrally located and joined to tabbed wire members 18 and 20 by conductive, two-sided, tape members 16. Above and below the PVDF member 14, conductive tape 16, and wire lead assembly are double sided adhesive foam members 30. The adhesive foam members 30 have a dogbone or dumbbell shape very similar to that of PVDF film member 14. The foam members 30, however, have enlarged heads at their ends which are slightly longer than those of the PVDF member 14. Such additional length allows the PVDF film member 14 to be fully contained within its confines. The foam members 30 have plural alignment points 32 where the perimeter of its center section and outer enlarged heads come together. The alignment points 32 for the foam member 30 correspond and match the dimensions of the alignment points 28 of the PVDF member 14. This common characteristic helps to ensure proper alignment of the various layers of the invention during assembly and manufacturing. The purpose of the foam member 30 is primarily to hold the PVDF film 14 and other components in place during assembly. Without such foam the PVDF film 14 is especially difficult to position and align due to its thin and hard to handle surface.

FIG. 4 also shows a pair of elastic urethane members 34 and 34′ which encompass the sensor member assembly to serve as a single unitary polyether polyurethane envelope to seal the PVDF film member 14 and its assembly from moisture. The urethane members 34 and 34′ are largely rectangular in shape and slightly larger than the PVDF film member 14. Because the urethane members 34 and 34′ are slightly larger in their dimension than the PVDF film member 14 and foam members 30, the PVDF film assembly can be contained entirely within the confines of the urethane members' dimensions. Further, the urethane members 34 and 34′ can be easily aligned during manufacturing by matching the notches 36 with the corresponding alignment points 28 and 32 of the PVDF and foam members.

Once aligned, the urethane members 34 and 34′ are sealed around the perimeter of film 14, foam 30, and wire leads 18 and 20 by an impulse sealing procedure. In such an impulse sealing operation, temperature and pressure are applied to encase the interior assembly. The resulting assembly is an airtight configuration that is resistant to moisture and corrosive substances that might harm the PVDF film member 14

Assembly of this embodiment is completed when the urethane envelope is aligned between a label and the surface of the belt member 12 and these three elements are joined with one another. In this embodiment, such attachment is preferably performed by stitching the ends of the members to the belt 12, but other attachment means can be employed. The stitching 38 generally extends laterally across the width of the belt and sensor assembly at a location corresponding to a second pair of notches 40 at both ends of the urethane layers 34 and 34′. Placing the stitching 38 in this location ensures that the stitches will pass through the ends of the foam members 30, but are just beyond the ends of the PVDF material 14 to avoid any damage to the film. All these members are sized such that when aligned, precise stitching and assembly can take place. The resulting assembled belt has an outside appearance similar to that of FIG. 3.

Those skilled in the art will appreciate that the belt sensor of the present invention may be manufactured in a variety of shapes and sizes to accommodate various sizes and types of persons and belt configurations. The components can be composed of any number of suitable materials. Also, the design of the present invention should not be construed to limit its application to only respiratory sensing applications.

The invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

1. A respiratory sensing belt comprising:
(a) a film member having piezoelectric properties and first and second metalized major surfaces wherein the surfaces are of symmetric shape and contain a plurality of spaced apart alignment features around the surface perimeter;
(b) a first tabbed lead and a second tabbed lead respectively attached to the first and second metalized major surfaces of the film member by an electrically conductive adhesive;
(c) a label for concealing the film member; and
(d) a body encircling elastic belt member wherein the film member is affixed to the surface of the belt member such that stretching of the belt member produces an electrical signal between the first and second tabbed leads.

2. The respiratory sensing belt as in claim 1 wherein the film member is polyvinylidene fluoride.

3. The respiratory sensing belt as in claim 2 wherein the film member has an elongated rectangular portion.

4. The respiratory sensing belt as in claim 3 wherein the film member has an enlarged head at both ends.

5. The respiratory sensing belt as in claim 1 wherein the film member has a dumbbell shaped perimeter.

6. The respiratory sensing belt as in claim 5 wherein the body encircling belt member is adapted to fit around the abdomen of a user.

7. The respiratory sensing belt as in claim 5 wherein the body encircling belt member is adapted to fit around the chest of a user.

8. The respiratory sensing belt as in claim 1 wherein the respiratory sensing belt is disposable.

9. The respiratory sensing belt as in claim 1 wherein adhesive foam members are placed around the film member and tabbed leads.

10. The respiratory sensing belt as in claim 9 wherein a urethane member forms a seal around the outside of the foam members.

11. The respiratory sensing belt as in claim 10 wherein the label, the foam members, and urethane layers are stitched to the belt member.

12. The respiratory sensing belt of claim 11 wherein the respiratory sensing belt is reuseable.

13. A respiratory sensing belt comprising:
(a) a film member having piezoelectric properties and first and second metalized major surfaces;
(b) a first tabbed lead and a second tabbed lead respectively attached to the first and second major surfaces of the film member by an electrically conductive adhesive;
(c) a pair of foam members sandwiching the film member and tabbed leads therebetween;
(d) a urethane member surrounding the foam members in a moisture resistant configuration;
(e) a label concealing the film member; and
(f) a body encircling elastic belt member wherein the assembly elements (a)-(e) is affixed to the surface of the belt member.

14. The respiratory sensing belt of claim 13 wherein the film member, the foam members, and urethane coating have corresponding features in their shape for alignment.

15. The respiratory sensing belt of claim 14 wherein said assembly is stitched to the belt.

16. The respiratory sensing belt of claim 15 wherein the respiratory sensing belt is reusable.

17. A respiratory sensing belt comprising:
(a) a body encircling belt member;
(b) a sensor member affixed to the belt member by stitching wherein said sensor member comprises a symmetrically shaped PVDF film having two major surfaces that are metalized and having a generally rectangular shape with enlarged heads at both ends, an electrically conductive adhesive material coupled to the top and bottom major surfaces of the enlarged head of the PVDF film, a plurality of tabbed leads coupled to the metalized surfaces by electrically conductive adhesive material, a pair of foam members surrounding the tabbed leads and PVDF film, and a stretchable elastomeric envelope surrounding the foam members and a label affixed to the belt and covering the envelope.

18. The respiratory sensing belt of claim 17 wherein the PVDF film, foam member, and elastomeric envelope have corresponding features on their perimeter for aligning the assembly.
